(19) United States
(12) Patent Application Publication
(10) Pub. No.: US 2006/0089623 A1
(43) Pub. Date: Apr. 27, 2006

(54) Surgical instrument with wear-resistant housing and method of operating same

(75) Inventors: Durrell G. Tidwell, Burleson, TX (US); David Allen Goodrich, Grapevine, TX (US); Brian George Barber, Southlake, TX (US); Jason T. Franz, Austin, TX (US)

Correspondence Address: HAYNES AND BOONE, LLP, 901 MAIN ST, SUITE 3100, DALLAS, TX, 75202, US

(73) Assignee: Medtronic, Inc., Minneapolis, MN (US)

(21) Appl. No.: 10/970,546

(22) Filed: Oct. 21, 2004

(51) Int. Cl. A61B 17/00

(52) U.S. Cl. 606/1

(57) ABSTRACT

A surgical instrument and a method according to which a vane is disposed in a rotor housing and is rotated by the impingement of air on the vane to drive a shaft to which a surgical tool is attached. The housing is fabricated of a relatively hard material to reduce the wear on it when compared to other materials.

### FIELD OF THE INVENTION

The present invention relates generally to a surgical instrument, and a method of operating same, for the purpose of cutting and dissecting bone and other tissue.

### BACKGROUND

Many conventional surgical instruments employ pneumatic motors to rotate a cutting element for performing surgical procedures, such as the dissection of bone or other tissue. These types of motors include a series of vanes that are rotated at a relatively high speed in a rotor housing in response to the passage of high pressure air through the housing, to drive a rotary shaft. A cutting or dissection tool, or the like, is coupled to the shaft for rotation with the shaft at the relatively high speed for use in the surgical procedure.

The above-mentioned vanes seal against the inner wall of the housing which is often fabricated from a material, such as stainless steel or cast iron. As a result of the high-speed rotation, the vanes tend to wear down the inner diameter of the housing which, over time, compromises the seal and therefore the operation of the motor.

In order to overcome this problem, the inner surface of the rotor housing has been coated with a relatively hard material. However, since the material below the surface of the coating is not as hard, the coating tends to score and flake off with use, which exacerbates the problem.

All patents listed in Table 1 are hereby incorporated by reference herein in their respective entities. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the teachings of the present invention.

TABLE 1

| Patent/Publication No. | Patented/Published Date | Inventor |
|---|---|---|
| 4,068,987 | Jan. 17, 1978 | Crooks |
| 4,197,061 | Apr. 08, 1980 | Hill |
| 5,834,870 | Nov. 10, 1998 | Tokushima et al. |
| 6,413,062 | Jul. 02, 2002 | Peters |
| 2002/0151902 A1 | Oct. 17, 2002 | Riedel et al. |
| 2003/0023256 A1 | Jan. 30, 2003 | Estes et al. |
| 2003/0163134 A1 | Aug. 28, 2003 | Riedel et al. |
| 6,626,577 | Sep. 30, 2003 | Horng, et al. |
| 2003/0229351 A1 | Dec. 11, 2003 | Tidwell et al. |

### SUMMARY

According to an embodiment of the invention, the wear resistance of the rotor housing of the pneumatic motor is increased significantly when compared to prior art designs, thus enabling a satisfactory seal between it and the vanes to be maintained over a relatively long period of time.

Various embodiments of the invention discussed below may possess one or more of the above features and advantages, or provide one or more solutions to the above problems existing in the prior art.

### BRIEF DESCRIPTION OF THE DRAWINGS

FIG. 1 is an isometric view of a surgical instrument according to an embodiment of the present invention.

FIG. 2 is an enlarged exploded view of the instrument of FIG. 1.

FIG. 3 is an enlarged, partial sectional view of the embodiment of FIGS. 1 and 2 shown in an assembled condition.

FIG. 4 is an end view of the components of the instrument of FIG. 4.

### DETAILED DESCRIPTION OF THE PREFERRED EMBODIMENTS

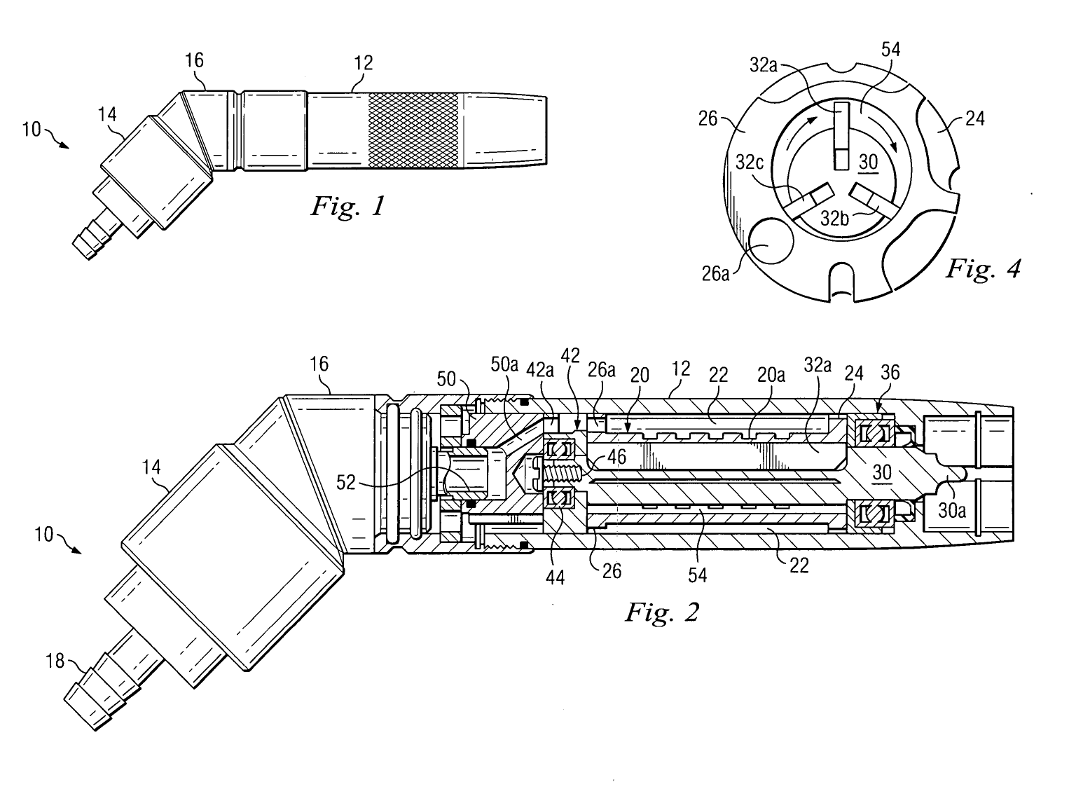

Referring to FIGS. 1 and 2 of the drawings, the reference 10 refers, in general, to a surgical instrument according to an embodiment of the invention which includes an outer casing 12 connected to a swivel assembly 14, via a coupler 16. One end of the coupler 16 is in threaded engagement with the rear end of the casing 12 and the other end is connected to one end of the assembly 14 by a conventional swivel connection which will not be disclosed in detail.

An air inlet tube 18 has one end portion projecting from the other end of the assembly 14 for attachment to an air hose (not shown), so that air passes through the assembly 14 and the coupler 16 to the interior of the casing for use in a manner to be described. The front end of the casing 12 is open and is adapted to receive a cutting element (not shown), a portion of which would extend in the casing for connection to the instrument 10 in a manner to be described.

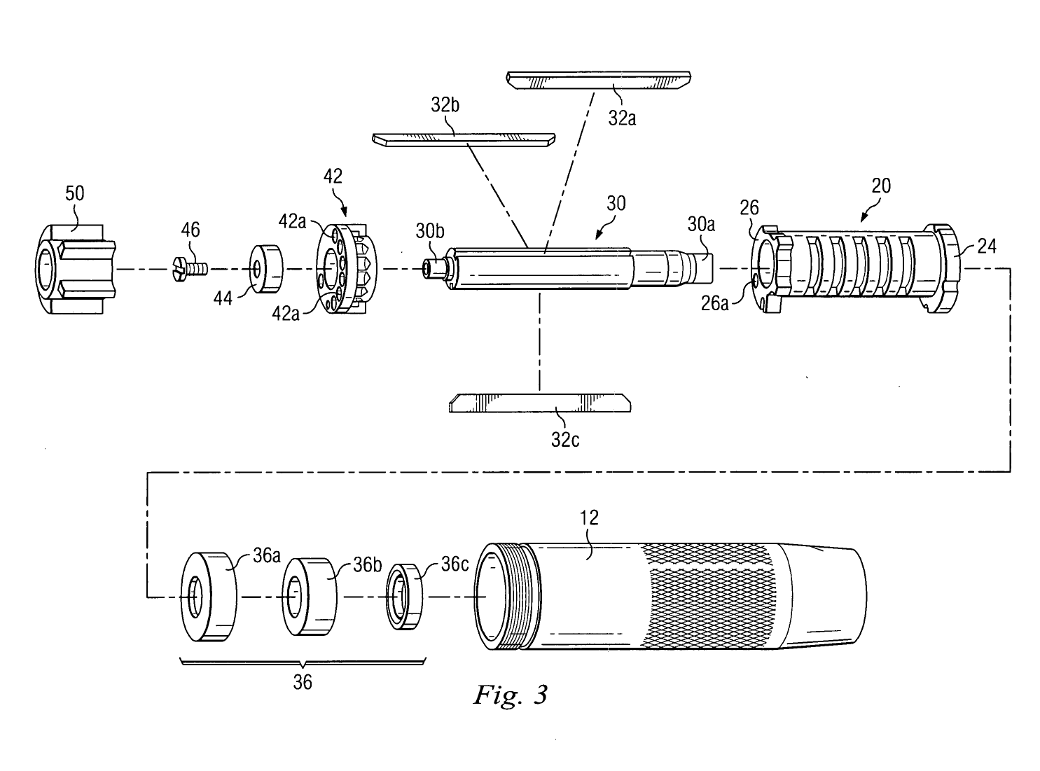

Referring to FIGS. 2 and 3, a cylindrical rotor housing 20 is located in the casing 12 with the outer surface of the housing extending in a spaced relation to the inner surface of the casing 12 to define an air chamber 22. The rotor housing 20 is fabricated from a carbide material for reasons to be described.

Two annular flanges 24 and 26 are formed at the respective ends of the housing 20, and a through opening 26a is formed in the flange 26 for reasons to be described.

The outer diameter of the flanges correspond to the inner diameter of the casing 12 so that the outer surfaces of the flanges engage the inner wall of the casing with minimal clearance to support the rotor housing 20 in the casing. A series of five spaced, parallel arcuate air slots 20a are formed in the housing 20 for permitting the ingress of air into the interior of the housing under conditions to be described.

A shaft 30 is supported in the casing 12 in a manner to be described, and a mounting flange 30a is formed at one end of the shaft 30 that projects from the corresponding end of the housing and is adapted to be engaged by the above-mentioned cutting element (not shown). A reduced-diameter portion 30b is formed at the other end of the shaft 30 and projects out from the other end of the housing 20 for reasons to be described.

Three elongated vanes 32a, 32b and 32c are disposed in three angularly-spaced, longitudinal slots formed in the outer surface of the shaft 30. Portions of the vanes project from the slots and the vanes are adapted for radial movement in the slots under conditions to be described.

A bearing assembly 36 extends in the casing 12 and around the front end portion of the shaft 30. The bearing assembly 36 is conventional and, as such, consists of a housing 36a, a bearing 36b that extends in the housing, and a seal 36c. As shown in FIG. 2, the bearing housing 36a and the bearing 36b are located between the front surface of the flange 24 and a shoulder formed in the interior of the casing 12, and the seal 36c extends in a groove formed in the casing and engages the bearing 36b.

A bearing assembly 40 is also disposed in the casing 12 and extends around the reduced-diameter portion 30b of the shaft 30. The bearing assembly 40 is conventional and, as such, consists of a housing 42 (FIG. 3) and a bearing 44 that extends in the housing. A series of angularly-spaced, through openings 42a are provided through the housing 42, for reasons to be described. A set screw 46 threadedly engages a threaded opening in the reduced-diameter portion 30b of the shaft 30, with its head engaging the bearing 44 to maintain the assembly 40 in the above position.

The shaft 30 is thus supported for rotation in the casing 12 by the bearing assemblies 36 and 40, with the mounting flange 30a of the shaft 30 located in the interior of the front end portion of the casing 12 so that it can be coupled to a standard cutting tool (not shown) in a conventional manner. Thus, when the shaft 30 is rotated in a manner to be described, it drives the tool.

An annular air distributor 50 is disposed in the casing 12 between the bearing assembly 40 and the rear end of the casing. A tube 52 (FIG. 2) extends from the assembly 14 and through the coupler 16 into a central opening in the distributor 50. Thus, air from the assembly 14 (FIG. 1) is passed, via the tube 46, to the distributor 50.

As shown in FIG. 2, an internal air passage 50a is provided in the distributor 50 that connects the air tube 46 to one of the openings 42a of the bearing housing 42. The latter opening is in alignment with the opening 26a in the flange 26 of the housing 20 so that the air passes from the tube 46, through the passage 50a, the openings 42a and 26a, and into the air chamber 22.

As shown in FIGS. 2 and 4, the shaft 30 is eccentrically disposed in the housing 20 to define an annular chamber 54 that varies in thickness, or cross section, in an angular direction around the shaft. Thus, as the vanes 32a, 32b, and 32c rotate with the shaft 30 under conditions to be described, the vanes move radially in the above-mentioned slots in the shaft 30 depending on their angular position in the chamber 52.

In operation, a tool is coupled to the mounting flange 30a of the shaft 30 and an air hose is connected to the tube 18 of the assembly 14. The air passes through the later assembly, through the tube 52, the passage 50a, the openings 42a and 26a, and into the air chamber 22. From the chamber 22, the air passes through the slots 20a in the housing 20 and into the chamber 54 where it impinges against the vanes 32a, 32b, and 32c, causing rotation of the shaft 30 to drive the above-mentioned cutting tool. During this action, the vanes 32a, 32b, and 32c are pushed, or forced, radially outwardly against the inner wall of the housing 20 as they rotate with the shaft 30, thus establishing a seal between the outer surfaces of the vanes and the inner wall of the housing 20. This also causes wear on the inner wall of the housing 20, but this wear is minimized by the relatively hard, carbide rotor housing and is relatively low when compared to prior art designs utilizing housings of a different, less hard, material.

It is understood that the motor 10, including the interface between the vanes and the inner wall of the rotor housing 20, can be lubricated in accordance with conventional techniques.

It is understood that variations may be made in the above without departing from the scope of the invention. For example, the number of vanes, as well as the structure for introducing air into the casing 12 and/or into the housing 20, may be varied. Further, the shaft 30 can be used to drive any type of surgical tool. Moreover, the rotor housing can be self lubricating as disclosed in co-pending U.S. application Ser. No. ______ (attorneys docket P-21152.00), the disclosure of which is incorporated by reference. Also, the specific type of motor used is not limited to a pneumatic motor.

The preceding specific embodiment is illustrative of the practice of the invention. It is to be understood that other expedients known to those skilled in the art or disclosed herein, may be employed without departing from the invention or the scope of the appended claims. For example, the present invention is not limited to surgical instruments, but may find further applications in which high speed rotation of a relatively small motor is required.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. A surgical instrument comprising a shaft, a housing having a cylindrical wall extending around the shaft in a spaced relation to the shaft for defining a chamber, and at least one vane extending from the shaft so that air introduced into the chamber impinges on the vane and rotates the shaft, the housing being fabricated from a carbide material.

2. The instrument of claim 1 wherein the air forces the vane against the inner wall of the housing to establish a seal, and wherein the carbide material reduces wear on the inner wall when compared to other materials.

3. The instrument of claim 1 wherein the housing is mounted in a casing, and wherein the air is introduced into a space defined between the housing and the casing and into the chamber through slots formed in the housing.

4. The instrument of claim 1 wherein there are three angularly spaced vanes extending between the shaft and the housing.

5. The instrument of claim 1 wherein a portion of the vane extends in a slot formed in the shaft and the shaft is eccentrically disposed in the housing so that the vane moves radially in the slot during the rotation of the shaft.

6. The instrument of claim 1 wherein the shaft is adapted to be connected to a tool for performing a surgical procedure.

7. A method of operating and lubricating a surgical instrument comprising fabricating a rotor housing of a carbide material, mounting a vane in the housing for rotation, and impinging air against the vane to rotate the vane and force the vane against the inner wall of the housing to establish a seal, the carbide material reducing wear on the inner wall when compared to other materials.

8. The method of claim 10 further comprising connecting the shaft to a tool for performing a surgical procedure.

9. The method of claim 7 further comprising mounting the housing in a casing, and wherein the air is introduced into a space defined between the housing and the casing and into the chamber through slots formed in the housing.

10. The method of claim 7 further comprising mounting a shaft in the housing for rotation with the vane and wherein the step of impinging comprises introducing the air into a chamber defined between the shaft and the housing.

11. The method of claim 10 further comprising disposing a portion of the vane in a slot in the shaft and mounting the shaft eccentrically in the housing so that the vane moves radially in the slot during the rotation of the shaft.
